(19) United States
(12) Patent Application Publication
(10) Pub. No.: US 2012/0013849 A1
(43) Pub. Date: Jan. 19, 2012

(54) Apparatus and method of monitoring and measurement using spectral low coherence interferometry

(75) Inventors: Adrian Podoleanu, Kent (GB); Michael Leitner, Linz (AT)

(73) Assignee: UNIVERSITY OF KENT AT CANTERBURY, Kent (GB)

(21) Appl. No.: 12/835,221

(22) Filed: Jul. 13, 2010

(51) Int. Cl. A61B 3/10; G01B 11/02

(52) U.S. Cl. 351/221; 356/496

(57) ABSTRACT

A spectral interferometry apparatus and method are disclosed, that can be used to monitor or measure an unknown length by following a characteristic of an indicating signal. The measurement is performed by adjusting an optical path difference (OPD) in an interferometer until sound or light or both are obtained with the desired strength and pitch. Embodiments are presented where the unknown length is the eye length. Sound of different pitches are produced by scanning the channeled spectrum output of an interferometer with the object returning at least one of the interferometer optical signals. The scanning is performed by reading the signal of an analogue photodetector array driven by a nonlinear clock or by tuning a low cost swept source using a distorted driving signal.

### TECHNICAL FIELD OF THE INVENTION

The present invention relates to a spectral interferometry apparatus and method, which can be used to monitor or measure an optical path difference by following a characteristic of an indicating signal.

### BACKGROUND OF THE INVENTION

There is a growing interest in the application of low coherence interferometry in the general field of sensing. Low coherence interferometry methods provide absolute distance measurements and are well suited for measuring absolute or relative distances based on signal returned by rough reflecting surfaces. Spectral low coherence interferometry (LCI) methods are based on the measurement of periodicity of the channeled spectrum of the optical signal coming from a two beam interferometer. The larger the optical path difference (OPD) in the interferometer, the denser the spectral modulation of the channeled spectrum. This can be read using a spectrometer, employing a dispersing element, such as a prism or a diffraction grating, to disperse respectively diffract light on a linear photodetecting camera to transduce the channeled spectrum of the interferometer output into a temporal signal, when the interferometer is excited by a large bandwidth optical source. Alternatively, a narrow band tuneable optical source, a swept source (SS), can be employed. By tuning the optical frequency of the optical source, the channeled spectrum is scanned point by point and a temporal signal is obtained again.

Channeled spectrum methods have been used in the sensing and fibre optic sensing field. Several implementations are known, using photodetector linear arrays, such as CCD and CMOS, to interrogate the optical signal output of the sensing interferometer, which allows to scan the channeled spectrum and produce a measuring signal. Such a method and device are disclosed in “Channeled Spectrum Display using a CCD Array for Student Laboratory Demonstrations”, published by A. Gh. Podoleanu, S. Taplin, D. J. Webb and D. A. Jackson in the European J. Phys., 15, (1994), p. 266-271.

The advantage of the spectral methods is that the OPD information is translated into the periodicity of peaks and troughs in the channeled spectrum and no mechanical means are needed to scan the object in depth, when performing optical coherence tomography (OCT) of tissue.

If multi-layered objects are imaged, such as tissue, each layer will imprint its own channeled spectrum periodicity, depending on its depth, with the amplitude of the spectrum modulation proportional to the square root of the reflectivity of that layer. A fast Fourier transform (FFT) of the signal delivered by a linear photodetector array, a CMOS or CCD linear camera signal, translates the periodicity of the channeled spectrum into peaks of different frequencies, with the frequency directly related to the OPD value. This measurement method is called frequency domain LCI (FD-LCI). The reflectivity profile with depth obtained by FFT is termed as an A-scan. Grouping together several A-scans, a B-scan or a cross section optical coherence tomography (OCT) image is obtained.

If a SS is used to scan the channeled spectrum of the interferometer, then the channeled spectrum profile is obtained directly in time, as a signal delivered by a photodetector device, method called SS-LCI. The FFT of such a signal leads to an A-scan again.

The methods above present the disadvantage that information is obtained by performing FFT. This requires a processor or a PC. Also, the standard method requires a display device, usually a monitor of a PC or a Laptop. Despite the continuous progress in computing and digital signal processing, these systems and devices raise the size and cost of FD-LCI and SS-LCI systems and of their OCT counterparts, FD-OCT and SS-OCT systems.

In measurements of distances in the field, in constructions, industry, portable systems are required. To extend spectral domain—LCI measurements to such sensing and industrial applications, low cost, small size and reduced weight systems are necessary.

In ophthalmology, measurement of eye length is performed before any cataract operation. Such measurements are performed using high cost instruments. Such instruments have a large size and are expensive. There is a need for such measurements to be more accessible to small ophthalmology practices. There is also a proven need to liaise the audio signal to the value of a quantity to be measured in complex environments where the sight is concentrated on the most complex tasks, such as surgery.

The patent application US2005/023727 A1, by Podoleanu and Rogers, used a loudspeaker to indicate the strength of the interference signal in a time domain optical coherence tomography system. The audible signal strength was an indication of signal detected and was not used in any measurement of any quantity.

Patent application US2008/0218588 A1, proposes an audio signal to transmit information about a captured image. However, this audio signal is used for transmission means only and does not allow for the direct monitoring or measurement of a system parameter.

The present invention provides methods and apparatuses which can advantageously perform measurements of lengths using a minimum of devices which can be conveniently assembled in a small size, low weight and low cost instrument that can be operated independent of computational power, simply by following a meter indication, a needle, a digital indication, a source of light or a source of sound.

### SUMMARY OF THE INVENTION

According to a first aspect of the invention there is provided a spectral interferometry method to measure an unknown length, based on an interferometer where an adjustment of an adjusting length device is performed until sound of a certain frequency is obtained. When the sound reaches maximum intensity, the value of the adjusting length provides a measure of the unknown length.

The unknown length could be that between an object and the instrument, could be that between two reflectors in a sensor, between two walls in constructions, between two parts in robotics, or the distance between the cornea and retina in an eye.

The present invention relates to a spectral interferometry apparatus and method, which can be used to monitor or measure an optical path difference by following a characteristic of an indicating signal.

In particular, the method may be Fourier domain optical low coherence interferometry (FD-LCI) or swept source optical low coherence interferometry (SS-LCI). As a further particularity, the characteristic of the indicating signal is the strength of sound and/or its pitch, or the strength of signal of a certain frequency passing through a pass band filter and determining an indication in the form of a voltage, light or sound. According to the invention, the measurement and monitoring may be performed without resorting to digital display or computational power, no PC is necessary. The method and apparatus presented may require a single adjustment until maximum is achieved for a sound or for the indication shown by a needle meter or by a digital meter, or for the intensity of light emitted by a displaying unit or by a light emitting diode (LED) or by several such LEDs. The adjustment may involve rotation of a knob or sliding a cursor along a ruler which reads the value of the unknown length. Guidance on the adjustment direction of the knob or cursor may be provided by following the pitch of the sound. Guidance on the adjustment direction may also be provided by the colour of a displaying device.

In a second aspect, there is provided a spectral interferometry apparatus using a broadband source and a linear array in a spectrometer which provides the measuring signal.

In a third aspect, there is provided a spectral interferometry apparatus where a tunable narrow band source and a photodetector unit delivers the measuring signal.

In a fourth aspect, measurement of the eye length is performed by focusing light on the anterior chamber, collecting light from the anterior chamber via a first optical delay and collecting light from the retina along a second optical delay, and where the measurement consists in adjusting one of the first or second delay or both until maximising the indication of a meter or the intensity of a light source or the strength of a sound of a certain frequency. Embodiments are disclosed based either on a large band source and a linear camera, or based on a tuneable narrow band source and a photodetecting unit.

In a fifth aspect, a configuration of Talbot bands is implemented to shift the maximum sensitivity of the FD-LCI method away from zero OPD, to a value of reference, OPD_{ref} and where the measurement of the unknown length is concluded when the OPD is adjusted to OPD_{ref}.

In a sixth aspect, a tandem interferometer is used, where a device is employed to modify an adjusting length in an adjusting interferometer part of the instrument and the unknown length is part of lengths of arms forming a sensing interferometer, where for example, the unknown length is the eye length, and where the beating of the two measuring signals, corresponding to each interferometer is produced using an electric mixer.

In a seventh aspect, a method and apparatus are provided to perform the operation of a stethoscope to produce sound at the rate of a heart beat, where the indicating signal follows in one or more of its characteristics, the movement of a heart wall,

The novel features which are believed to be characteristic of the present invention, as to its structure, organization, use and method of operation, together with further objectives and advantages thereof, will be better understood from the following drawings in which presently preferred embodiments of the invention will now be illustrated by way of example.

It is expressly understood, however, that the drawings are for the purpose of illustration and description only and are not intended as a definition of the limits of the invention.

### BRIEF DESCRIPTION OF THE DRAWINGS

Embodiments of this invention will now be described by way of example in association with the accompanying drawings in which:

FIG. 1 shows a block diagram of an apparatus according to the invention.

FIG. 2 shows a detailed embodiment of the invention using FD-LCI

FIG. 3A displays the typical output of a linear camera in an FD-LCI based spectral interrogator showing repetitions in time of the channeled spectrum.

FIG. 3B displays the optimized output of the linear camera used to read the channeled spectrum.

FIG. 4 shows a detailed embodiment of the invention using SS-LCI

FIG. 5 shows the plot of length measurements of a varying length following the strength of sound in the embodiment in FIG. 2.

FIG. 6 displays a first version of an apparatus for measuring the eye length using FD-LCI according to the invention.

FIG. 7 exemplifies a second version of an apparatus for measuring the eye length using FD-LCI according to the invention.

FIG. 8 exemplifies a third version of an apparatus for measuring the eye length using FD-LCI according to the invention.

FIG. 9 shows an apparatus for measuring the eye length using SS-LCI according to the invention.

FIG. 10 shows an apparatus for measuring the thickness of an object using a common path probe head with SS-LCI interrogation according to the invention.

FIG. 11 shows an apparatus for measuring the thickness of an object using a common path probe head with FD-LCI interrogation according to the invention.

FIG. 12 illustrates the linearization of data from an analogue linear camera used in SS-LCI

FIG. 13 illustrates the linearization of data from a photodetector in a FD-LCI.

FIG. 14 shows an improved diagram for the electronic processing unit.

Components which are the same in the various figures have been designated by the same numerals for ease of understanding.

Where optical fibres are used, this is only as an example and it should be noted that a bulk implementation is equally feasible, in which case the respective elements using in-fibre components, are to be replaced by optical paths and the directional fibre couplers by bulk beam-splitters, in the form of plates or cubes. Likewise, where bulk components are used, they could equally be replaced by optical fibre components.

### DETAILED DESCRIPTION OF THE INVENTION

The novel features which are believed to be characteristic of the present invention, as to its structure, organization, use and method of operation, together with further objectives and advantages thereof, will be better understood from the following discussion.

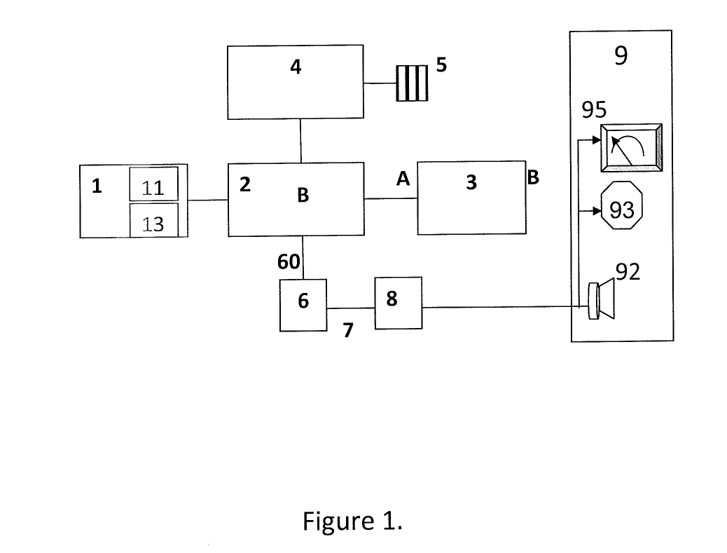

An embodiment of the apparatus according to the invention is shown in block diagram in FIG. 1, where light from an optical source block 1 is sent to an interferometer, 2. The OPD in this interferometer is determined, as the difference between a reference path length and an object path length. The object path length is measured from the interferometer, up to a point, point A on the object 3. The reference path length can be measured either along a reference path length inside the interferometer, or up to a point on the object, point B. In the first case, the apparatus is used to measure distance AB between the object, 3 and the interferometer, 2, in which case an arbitrary point B is considered inside the apparatus, shown for example inside block 2. In the second case, the apparatus is used to measure the thickness AB of the object 3. The OPD can be adjusted actuating on a translations stage, 4, equipped with measuring means, 5, in the form of a micrometer screw, a graded knob or a sliding ruler. Equivalently, the translation stage may be replaced by a spectral scanning delay line, using a diffraction grating, a focusing element and a tilting mirror according to means known in the art, and where the OPD is adjusted by tilting the mirror. The output optical signal, 60, is sent to a spectral interrogator, 6, producing a measuring signal 7, processed by an electronics processing unit, 8. This is delivered to a block of indicators, 9, which contains a loudspeaker, or an earphone 92, or a displaying unit, equipped with an LED or several LEDs of different colours, 93, or a needle indicator 95, such as a voltmeter or ammeter, or a digital meter. The optical source block includes a broadband optical source, 11, or could be a narrow band, tuneable source, SS, 13.

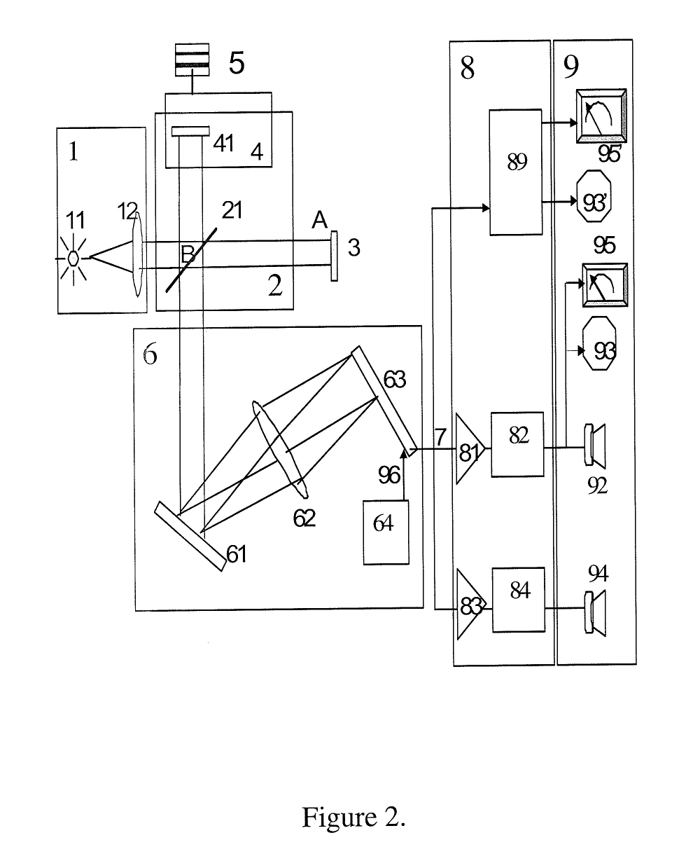

FIG. 2 shows details of an embodiment of apparatus according to the invention, where the optical source 1 consists in a broadband source, 11, such as a superluminiscent diode (SLD), a light emitting diode (LED), a thermal source, or any other optical source providing wide linewidth, such as photonic crystal fibre, and a collimating optical element, 12, in the form of a curved mirror or lens. The interferometer consists in a 1^{st} splitter, a beamsplitter 21, which splits light into two arms, a reference arm towards the mirror 41 in the adjusting length device 4, and an object arm towards the 1^{st} point, A, on the object 3. Here distance is monitored or measured between point A, on mirror 3 and beamsplitter 21, where the 2nd point, B is considered virtually situated inside the apparatus. Here again, the distance AB is measured from the object to the apparatus. Light beams returned from 3 and 41 are sent to the spectral interrogator, 6, which consists in a dispersing element, 61, in the form of a prism or diffraction grating, a focusing lens in the form of a curved minor or lens, 62, and a linear CCD or CMOS array, 63. Such devices have 512 to 4096 pixels, as an example only, and these pixels are read in sequence at a frequency F. The measuring signal 7 is sent towards the electronics processing unit, 8, which consists in one or more signal processing channels, two channels are shown for example only, equipped with amplifiers 81 and 83 and band pass filters 82 and 84, which drive the block of indicators 9, consisting in loudspeakers 92 and 94, display device 93 and/or voltmeter 95.

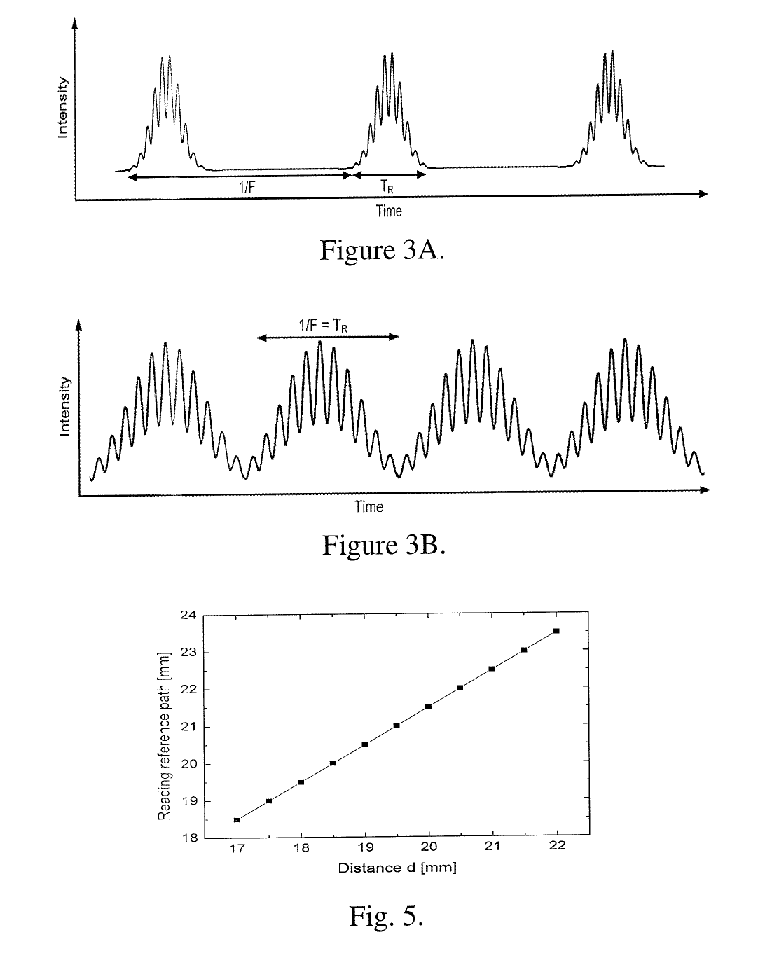

The signal 7 is fed out in a reading time T_{R}, which is less than 1/F usually, as shown in FIG. 3a. If such a signal is sent to a spectrum analyser, multiple components at frequencies F with side bands at 1/T_{R} are produced. According to the invention, for the application described here, it is important that the spectrum is as clean as much as possible, to allow provision of the useful information related to the OPD to be measured, deprived from stray frequency components. In order to clean the spectrum, the reading time, T_{R}, is adjusted to be as close as possible to 1/F. In this way, the signal output from the array looks like a smooth modulation with no interruption, as shown in FIG. 3b.

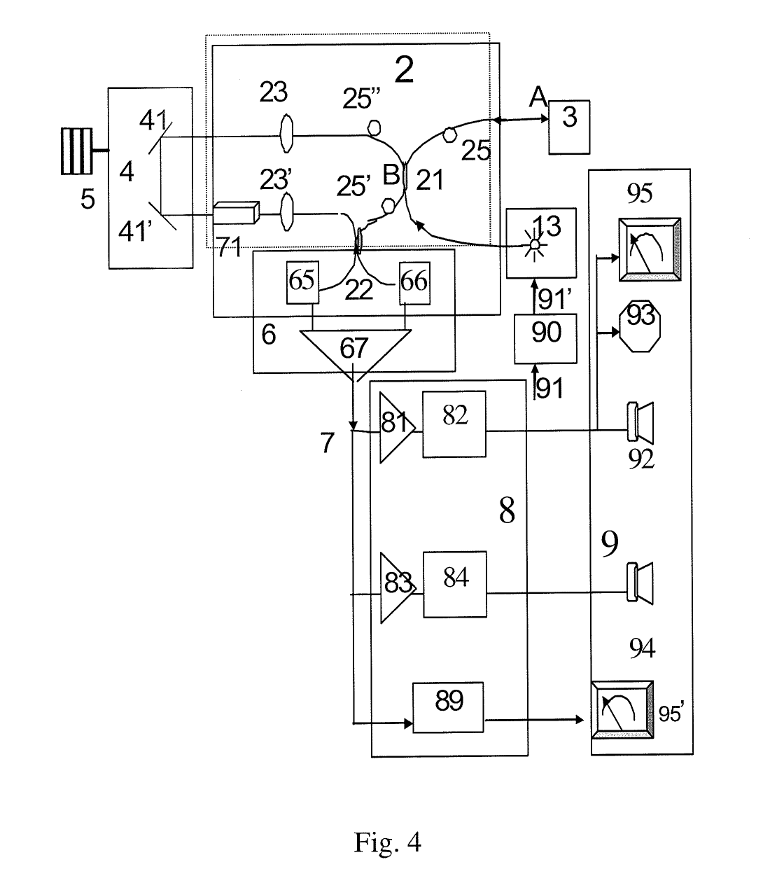

FIG. 4 illustrates another embodiment of the invention where principles of SS-LCI are used. In this case, the optical source block, 1, is made of a tuneable narrow band source, 13.

Those skilled in the art will recognise that different configurations are now known for optical sources to provide fast tuning rates of more than 100 kHz, of linewidth less than 0.1 nm within a bandwidth of more than 50 nm. Such sources use ring lasers equipped with an optical amplifier and an optical filter, as described in the following papers: M. A. Choma, K. Hsu J. A. Izatt, “Swept source optical coherence tomography using an all-fiber 1300-nm ring laser source,” J Biomedical Optics 10_{—}4, 044009_pp. 044009-1 to 044009-6, 2005 and R. Huber, M. Wojtkowski, and J. G. Fujimoto, “Fourier Domain Mode Locking (FDML): A new laser operating regime and applications for optical coherence tomography,” Opt. Express 14, pp. 3225-3237, 2006.

However, such sources are still very expensive and it is not generally desirable to pair a low cost optical configuration as presented in this disclosure with such expensive sources. For the purpose of the invention, lower cost sources are therefore preferred, such sources may be based on a semiconductor laser diode whose current is ramped. Such sources are known in the field of frequency modulation continuous wave (FMCW) sensing, as for instance used in the article “Reflectometric fiber optic frequency-modulated continuous-wave interferometric displacement sensor” published by Zheng, J in Optical Engineering, Vol. 44, Issue 12, Article Number: 124404, December 2005 In this way, a few nm tuning bandwidth is easily achievable. The small tuning bandwidth leads to a poor depth resolution. However, even depth resolutions worse than 50 microns could be tolerated for certain measurements in low cost solutions. Other low cost swept sources are being developed which could find applications in the invention, using micro-electro-mechanical (MEM) based tuneable resonators. The larger the tuning bandwidth the higher the cost of such sources. For digital processing, a Fabry-Perot interferometer is also incorporated into swept sources to provide a clock which is subsequently used for linearization of data. If tuning bandwidths of 5-10 nm are targeted and such sources are also simplified not to include clock generation and no other circuit for linearization, then their cost is dramatically reduced.

The spectral interrogator in FIG. 4 uses a photodetection unit, 6, equipped with a single photodetector or preferably, two photodetectors, 65 and 66 in a balanced detection configuration with a differential amplifier 67 that produces the difference of the two photodetected signals and delivers the measuring signal 7. This is then sent to the electronics processing unit 8. An in-fiber configuration is shown in FIG. 4, used to implement the interferometer 2. Light from the swept source, 13, is sent to a first splitter, 21, implemented here as an example using a single mode directional coupler which splits light into the object arm towards the point A, on the surface of object 3 and the reference arm, towards mirrors 41 and 41′ placed on the translation stage 4, using focusing elements 23 and 23′. Light from the object 3 is returned via 21 towards a balanced splitter, 22, implemented here as an example using a single mode directional coupler, where it interferes with light from the reference arm. The interferometer is equipped with means known in the art, to optimise the polarisation, 25, 25′ and 25″ and compensate for dispersion, such as optical slabs, 71.

Procedure

For both embodiments in FIGS. 2 and 4, a band pass filter, 82 is set on a frequency corresponding to a desired reference OPD value. For a central wavelength λ, spectral width Δλ, the axial range, from point A to point B is given by ΔZ=0.25Mλ²/Δλ, where M are the number of pixels in the CCD array in FIG. 2 or the number of resolvable frequency steps for the SS in FIG. 4. For a central wavelength of 800 nm, the equivalent coherence length for a source with Gaussian spectrum is l_{c}=0.44λ²/Δλ, this characterizes the depth resolution, i.e. the differential distance between adjacent sampling point values on the horizontal axis of the FFT. By equivalent coherence length l_{c} we mean here the coherence length of an equivalent time domain (TD)-LCI system excited by a broadband optical source 11 in FIG. 2 with spectrum width Δλ, equal to the tuning bandwidth Δλ, of the source 13 in FIG. 4. For example, for a Δλ=50 nm, l_{c}≅5.6 μm. The ratio ΔZ by l_{c} determines approximately the number of sampling points in the FFT, 0.25M/044˜M/2. For an OPD=l_{c}, the channeled spectrum exhibits one spectral modulation period and M=2 pixels are needed at least in the linear array 63 in FIG. 2 or at least M=2 frequency steps are required for the SS, 13, in FIG. 4.

Let us say that we choose to identify an unknown OPD=δZ, as a small part of the distance between the surface of the object 3, point A, up to the interferometer, point B. This would mean a δZ/l_{c} number of cycles in the channeled spectrum. Reading the linear array 63 in FIG. 2 at a frequency F, or tuning the frequency of the source 13 in FIG. 4 at a frequency F, will output a measuring signal, 7, of frequency f=F(δZ/l_{c}).

The Audio Signal can be Utilised in Two Ways:

Intensity

A band pass filter, 82, will only transfer signal to its output when the frequency of the input signal. 7, is that chosen for the process of monitoring or measurement. This reference frequency correspond to a chosen reference OPD value, δZ. Let us consider a readout CCD (or tuning) frequency F=1 kHz and δZ/l_{c}=10. This corresponds to a chosen reference value of the audio frequency f=10 kHz, as the main component in the frequency spectrum of the measuring signal. The measuring means 5 in FIG. 2 and FIG. 4 are adjusted to obtain a maximum for the signal at f in loudspeaker 92. The narrower the band of 82, the better the accuracy in the axial measurement. The maximum of sound heard in the loudspeaker 92 will indicate that the length of the OPD has reached the sought after reference value δZ and an indication of that length will be given by 5.

Pitch

Seeking maximum intensity in the loudspeaker requires scanning a knob or sliding a ruler in 5 in both directions. Preferably, the invention uses both the intensity of the signal at the output of 82 tuned on f as well as the pitch of the sound. To this goal, a second large bandwidth band pass filter, 84 is used. The pitch gives an indication on the direction of adjusting the measurement means 5. For the example above, the band pass filter 84 allows audio frequency signals of frequency 1 to 18 kHz, within the human hearing band.

The relative amplitude of the two signals in the two loudspeakers can be controlled by relatively adjusting the amplification in the amplifiers 81 and 83. When the signal entered into the bandwidth of 82, the signal in the loudspeaker 94 can be reduced to zero and the measurement finalised by maximising the sound in 92. Alternatively, only one loudspeaker is used, 94, to provide information on the direction of rotating the knob or sliding the cursor 5, and the optimum adjustment will only be guided by producing maximum in the light display device or LED 93 or/and the voltmeter 95. As a further possibility, several coloured LEDs are used, with different threshold actuating levels. A liquid crystal display device may also be used, or a coloured display device that display stripes of coloured bands, where the frequency of the colour is proportional to the amplitude of the signal. Several possibilities exist to sensitize the measurement, known being that the eye is more sensitive to colour difference than to the colour itself. Therefore, for each new position of the measuring means 5, the display device 93 provides at the same time, stripes of colour corresponding to the previous OPD value as to the current OPD value. The colour difference will then suggest the direction of adjusting the OPD using 5.

It is also possible to convert the frequency of the reading signal 7, into amplitude directly, using a frequency to amplitude convertor, 89, that drives a needle instrument, 95′, or a digital meter.

The measurement of OPD in both cases above relies on the value shown by the calibrated knob 5. This could be a micrometer screw, with division at 10 microns. Interpolation between such divisions can give a resolution in measurement better than 5 microns.

A proof of concept was set-up for the embodiment in FIG. 2 and using a band pass filter 82 tuned on 2.7 kHz with a bandwidth of 67.5 Hz was used. A mirror was used as an object, 3, placed on a stage whose distance was changed in steps of 0.5 mm. Then, by using the micrometer screw 5, the OPD was adjusted until maximum signal strength was obtained in the loudspeaker 92. The position was read on the scale of the micrometer screw 5 and the graph in FIG. 5 was obtained. This shows that simply following the sound in a loudspeaker and using a ruler, the distance from object 3 up to the interferometer can be measured and monitored with better than 10 micrometer accuracy.

Applications

The method according to the invention can be used for measurement as well as to monitoring of distances. The stage 4 can be set at a reference value and from that moment, the fluctuation in distance of the object mirror 3 can be evaluated by moving stage 4 until sound is regained in the loudspeaker 92. Automatic procedures can also be devised according to means known in the art, by using the signal towards loudspeaker 94. If the pitch sound is higher than the desired frequency f, by actuating on means 5, the stage 4 is moved in the direction of increasing the reference path, to reduce the OPD=object path−reference path. If the pitch is lower than f, then the stage 4 is moved to reduce the reference path length in the interferometer for example.

Eye Length Measurement

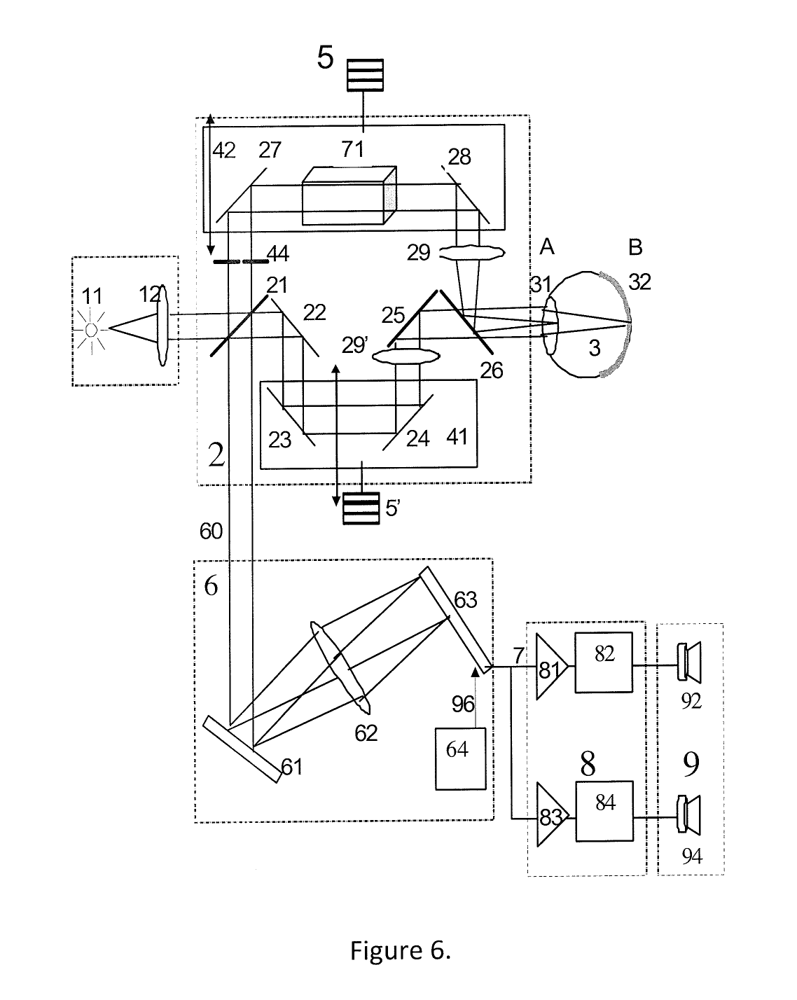

FIG. 6 illustrates an FD-LCI embodiment where the eye length of a patient, the distance between point A, on the cornea and point B, on the retina, is measured using the method according to the invention, based on sound monitoring or a meter indication or light produced. The output beam from the optical source, 1, is split into two beams by a first splitter, 21. The first beam, object beam, is reflected towards mirrors 27 and 28 placed on the translation stage 42, whose position is adjustable via the micrometer screw 5, part of measuring means, and then focused via an interface optics, shown in the form of a lens 29 and a second splitter, 26 on the cornea 31 of the eye 3. The second beam, reference beam, is sent to the retina, 32, of an eye, 3, via mirror 22, 23, 24, 25 and splitter 26. The mirrors 23 and 24 are placed on a translation stage, 41, which can be adjusted by micrometer screw 5′. To focus light on the retina, 32, the reference beam needs to be collimated for emmetropic eyes. For long sighted or short sighted eyes, a reference interface optics may be used to axially modify the focus position on the retina of the reference beam. A converging or diverging lens 29′ or a curved mirror, or an electrically controlled liquid crystal lens, or a deformable MEM mirror can be used as 29 and 29′.

A channeled spectrum is created by the interference of the two beams, object and reference reflected of the cornea 31, point A and from respectively retina 32, point B. The cornea signal is at least 1000 time stronger than the signal from the retina. Therefore, in order to balance the strengths of the two reflected signals, splitter 26 has a transmission much higher than the reflection, for instance 95% transmission (in which case a simple glass plate antireflection coated on one side could be used, or even 99%). Additional correction of amplitudes can be obtained by adjusting the transmission of the splitter 21 to smaller values than its reflection, in order to maximise the signal from the retina 32 for an input power towards the eye close to the safety limit. Further, an adjustable pinhole, 44, can be used to reduce the power towards the top, 31, of the object 3. In case the object is the eye, this also helps with extending the depth of focus of the object beam, to make the apparatus compatible with non-accurate axial distance position of the object 3 in respect to the apparatus.

One or both of the micrometer screws 5 or 5′ parts of the measuring means can be calibrated. The audio frequency of choice, 0.5-15 kHz can be chosen to correspond to a certain OPD value of reference, δz. This could be associated to the minimum, or the maximum, or the middle value in the range of eye lengths, let us say, to a value E=23 mm between points A and B. To accommodate measurements of eyes shorter or longer than this value, the micrometers 5 (or 5′) are equipped with rulers graded from 17 to 29 mm. Adjusting 5 or 5′ to regain maximum strength in 92 guided by the pitch in 94 leads to the current eye length value, E. Obviously, because the linear photodetector array, 63, may have only 1000-2000 pixels, the axial range may be limited to 1-4 mm. Therefore, the procedure involves turning the knob of the calibrated micrometer screw 51, or 52 or both until sound is heard in 94 followed by enhancement of sound in 92. It is possible that position of knobs (cursors, micrometer screws) 5 and 5′ are such that the OPD value is out of the limited axial range. In this case, the adjusting knob 5 or 5′ is moved to one extremity and back until the highest pitch, 18 kHz is heard. From that moment, adjustment is made to bring the pitch of the sound to that corresponding to the reference value δz.

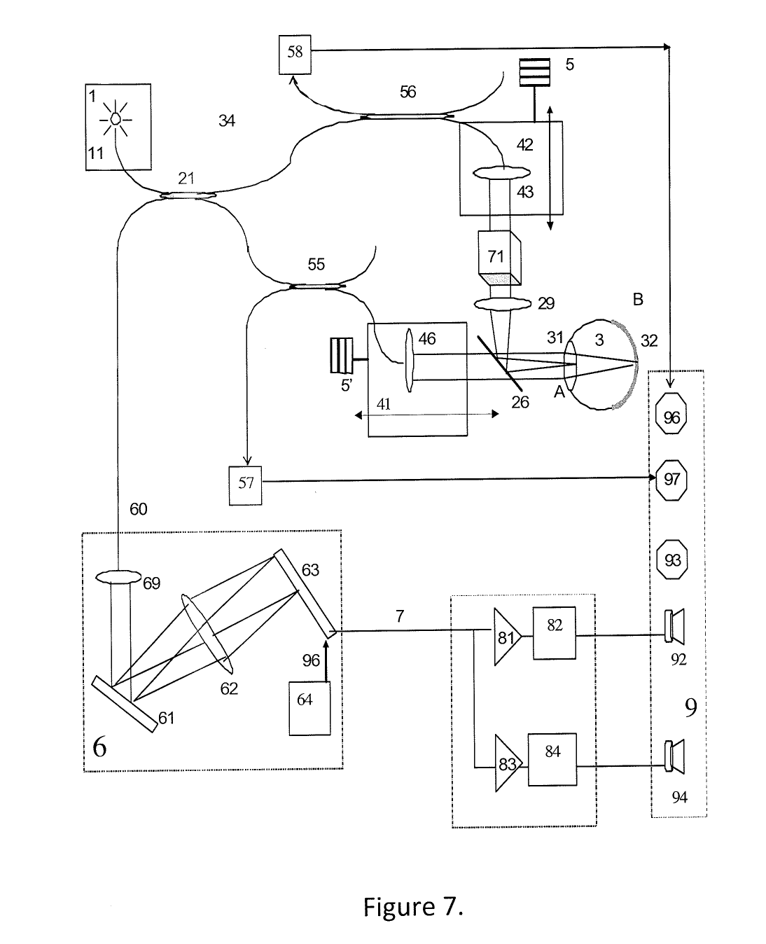

FIG. 7 shows an equivalent of the embodiment in FIG. 6, where the first optical splitter, 21, is a single mode directional coupler. The fiber output of 21 connects to the launchers situated on stages 41 and 42, via two couplers, 55 and 56. The positions of stages 41 and 42 are adjusted to bring the optical difference between the object path towards the cornea 31 and the reference path towards the retina, 32 to the reference value of the OPD, δz, which determines the sought after strength and pitch respectively in the loudspeakers 92 and 94. The two beams, object from point A, on the cornea 31 and reference beam from point B, on the retina, 32, are handled separately, as light form retina does not go through lens 32 and light from cornea does not go through lens 46. The object interface optics is formed from lenses 43 and 29 and aims to focus light on the cornea, 31. Lens 46 is adjusted to send a collimated beam towards the eye 3, in case the eye is emmetropic. For a short sighted or long sighted, the lens 46 can be moved in relation to the output fiber of splitter 21, to adjust the convergence of the reference beam launched into the eye 3, the fiber end and lens 46 are identified as reference interface optics. The output of coupler 21 provides the output optical signal 60, whose channeled spectrum is to be interrogated by the spectral interrogator 6. Optical signal 60 is launched as a collimated beam, via focusing element 69, towards the diffraction grating 61.

To tolerate eventual placements of the eye 3 away from the ideal axial position where the object beam focuses on the cornea 31, lens 43 has a small focal length to prepare a small diameter beam launched towards the lens 29, and lens 29 has a long focal length, and in this way, a long depth of focus is achieved. This advantageously leads to less efficiency in collecting backscattered light from the cornea, 31, which returns much more than the retina, 32.

As additional elements which can be carried to the other embodiments dealing with eye length measurement, two indicators, 96 and 97 are used in the indicating block 9, to inform the user that sufficient signal is returned from the point A on the cornea, 31 and from the point B, on the retina, 32. They could be LEDs, driven by photodetectors 57 and 58 respectively, at the output of single mode couplers 55 and 56 respectively. The two couplers 55 and 56 are used to tap small portions of the signals returned from retina and cornea to excite the photodetectors 57 and 58. Before any measurement, it may be necessary to adjust the convergence or divergence of the object and reference beams to enhance the strengths of the signals reflected from points A, 31 and B, 32. The adjustment can be performed by moving axially the lenses 29 and 29″, or by using liquid crystal, electrically controlled lenses 29 and 29″, or using deformable MEMS mirrors as 29 and 29″.

The process of measurement, consisting in actuating on the adjusting means 5, is performed only if LEDs 96 and 97 are lit up. The object 3, a slab or the eye, is adjusted laterally until sufficient signal is returned to the interferometer 2 from both points 1 and 2. Only then the OPD is adjusted towards the pitch sought after in the loudspeaker 94 and towards maximum sound in loudspeaker 92.

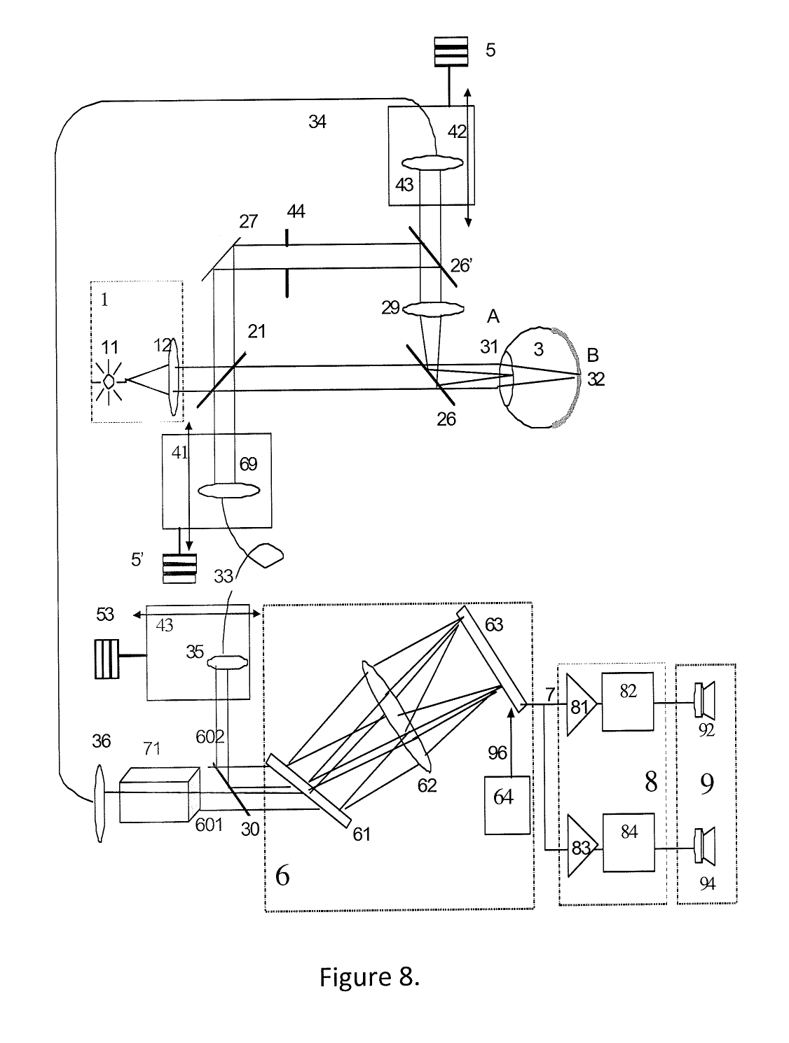

FIG. 8 shows another embodiment, based on Talbot bands, where the two beams from the anterior chamber and from the retina are sent along separate paths towards the diffraction grating. Light coming from the first splitter, 21, is deflected by a mirror 27, non-essential, towards the point A, on the surface of the cornea 31, via a third splitter, 26′ and second splitter 26. Light from cornea, point A, is split by splitters, 26 and 26′, into the fiber launcher on stage 42 equipped with focusing element 43, focusing light into fiber 34. This carries the light from the point A, on the cornea of eye 3, or from the anterior chamber towards lens 36 which launches a collimated beam, 601, towards the grating 61, via the optical splitter 30.

The light reflected from point B, the retina 32, traverses the optical splitters 26 and 21 towards the focusing element 69 in the fiber launcher placed on the stage 41, where light is launched into fiber 33. The two beams are separated, as light form point B, retina 32, does not go into fibre 34 and light from point A, cornea 31, does not go into fibre 33. Light from fibre 33 is then conveyed via focusing element 35 into a collimated beam, 602, via a fourth optical splitter, 30. The collimated beams 601 and 602 can be superposed or totally separated spatially by a gap using the translation stage 43 which moves the launcher with fiber end and focusing element 35 laterally, using the micrometer screw 53, as explained in the US patent application 2007/0165234, Spectral interferometry method and apparatus, by A. Podoleanu.

Fibers 33 and 34 have similar lengths to keep the dispersion low, along with the element 71, which compensates for the dispersion in the eye, usually a cuvette of approximate length equal to twice the eye length and filled with water. The procedure of measuring the eye length is similar to that described above where one of the stages 41, or 42 or both are driven by micrometer screws 5 and 5′. The path difference in air between the two beams from the anterior and posterior chamber of the eye are adjustable using the two stages 41 and 42. One of the micrometer screws (or both) is (are) equipped with a ruler showing eye lengths of 17-28 mm, to cover the normal range of eye length values.

The advantage of the Talbot configuration is that the maximum sensitivity of the FD-LCI method can be shifted from OPD=0 to a larger OPD value. The peak of sensitivity can be shifted to the value of OPD chosen as reference, δz, and that which gives the pitch of reference in the loudspeaker 92. The shift of peak of sensitivity from OPD=0 is proportional to the gap between the two beam 601 and 602 which can be adjusted using stage 43 and knob 53, that move the beam 602 parallel to beam 601 in its way towards 61.

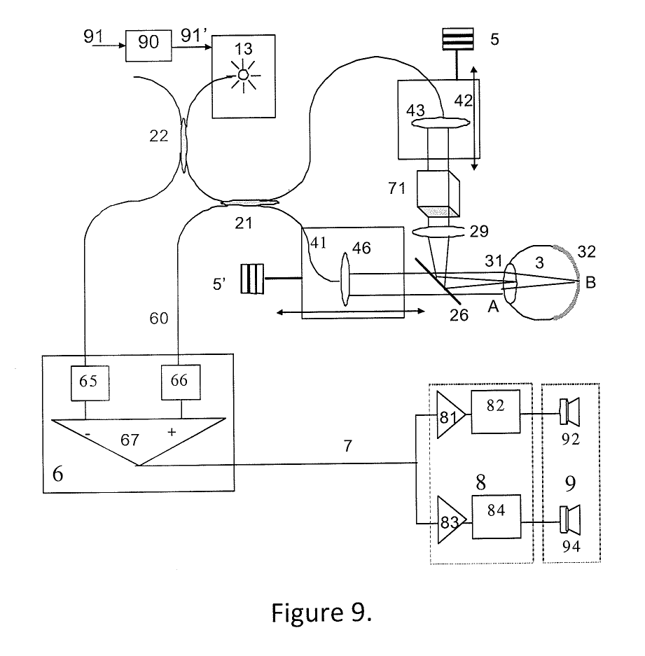

FIG. 9 discloses an embodiment for a similar application to that in FIG. 7, i.e. for measuring the thickness of object 3 or eye length of an eye 3, where the optical source is tuneable narrow band, 13. Balance detection is implemented using two directional couplers 21, as the first splitter and 22, as a fifth splitter, feeding two photodetectors, 65 and 66 whose photodetection currents are deducted in the differential amplifier 67. The two beams object, from point A on the surface of object 3, i.e. from cornea, 31, of an eye, and the reference beam from point B, retina, 32, are sent via separate paths. Light form retina does not go into focusing element 43 and light form cornea does not go into focusing element 46. The source 13 is tuned at a rate of 100 Hz for instance and for a reference optical path difference corresponding to 10 peaks in the channeled spectrum, the interference signal leads to an audio signal of 1 kHz.

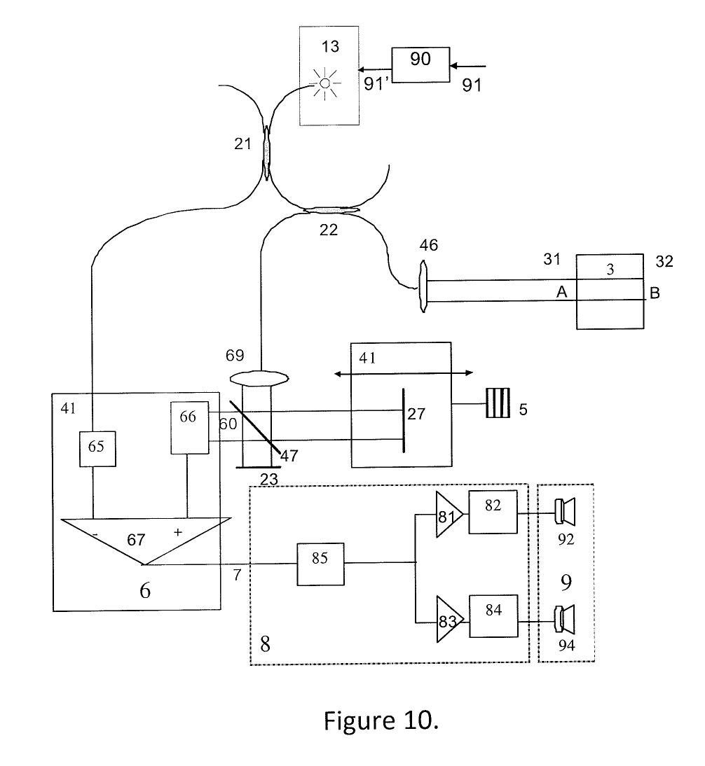

FIG. 10 discloses an embodiment where the thickness of the object, 3, a glass plate for instance subject to an external factor, is monitored using a tandem interferometer configuration. A first interferometer, sensing interferometer, is formed by reflections from the surfaces point A on surface 31, and from point B on surface 32 of the object 3, and a second interferometer, which is adjustable in OPD, is formed from the sixth splitter, 47, and mirrors 23 and 27. For each interferometer, a channeled spectrum modulation is produced. Let us say that the 1^{st} interferometer, of OPD₁ leads to a measuring signal 7 pulsating at a frequency f₁ when tuning the source 13. The adjustable interferometer, of OPD₂ determines a measuring signal 7 of frequency f₂ when tuning source 13. Using the knob 5 to alter OPD₂, the difference |OPD₁−OPD₂| is modified and consequently, the difference of frequencies |f₁−f₂|. Let us say that OPD₁ varies in time between OPD₁=Min=1 mm and Max=1.5 mm. OPD₂ can be adjusted in the range δz+Max−Min, where a frequency f is generated for a channeled spectrum corresponding to an OPD value δz. Let us say that the source 13 is tuned at a rate F=100 Hz and the channeled spectrum corresponding to Δ consists in 10 peaks. This means that a signal of frequency f=1 kHz is generated when |OPD₁−OPD₂|=δz. By adjusting the knob 5 to re-obtain the same pitch of 1 kHz in 94 allows monitoring of the OPD₁, value read from the calibrated ruler of knob 5. The frequencies f₁ and f₂ could be much larger than the audible range, for instance for OPDs corresponding to 1000 peaks, frequencies are over 100 kHz. These cannot be heard. Therefore, to produce the difference of frequencies, the block 85 is used. This operates like a mixer, producing nonlinear combinations of input signals, oscillating at multiple of frequencies nf₁+mf₂ where n and m could be any integer number. Block 85 may also contain high pass filters or band pass filters at its entry to reduce the noise and reduce the range of frequencies f₁ and f₂ applied to the mixer, as well as a low pass filter at its output, to eliminate the high frequency components.

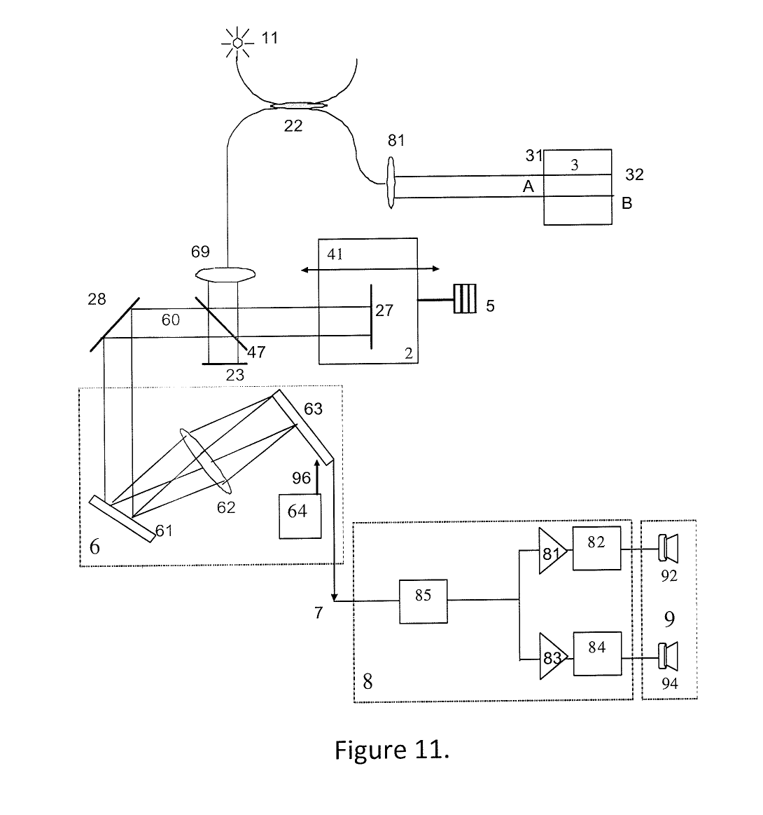

FIG. 11 discloses an equivalent embodiment to that shown in FIG. 10, this time using a broadband source, 11 and the principle of FD-LCI. A tandem interferometer configuration is used as in FIG. 10. The signal 7 is now delivered by the linear array, 63, with the repetition F as scanning the channeled spectrum, determined by the inverse of the integration time of the CMOS or CCD array used.

### Linearity

A problem for both FD-LCI in the embodiments in FIG. 2, 6, 7, 8, 11, as well as for the SS-LCI embodiments in FIG. 4, 9, 10, is that the AF spectrum of the signal delivered by the electronic processing unit, 8, is wide, unless the reading of the CCD (CMOS) array 63 and that of the photodetection units 65 (66) is linearised in relation to the optical frequency. Several methods have been proposed, where the data is digitised, zero-padded and only then a FFT is calculated. Such procedures are known for the person skilled in the art and can be implemented here as well if digital processing is adapted. FFT processors are now low cost and an initial calibration can instruct the software to be used. However this would involve an extra device and an extra procedure which may increase the cost.

As an inventive low cost solution, the present disclosure proposes a direct provision of a signal linear in optical frequency, when the measuring signal 7 is delivered in analogue format.

### SS-LCI

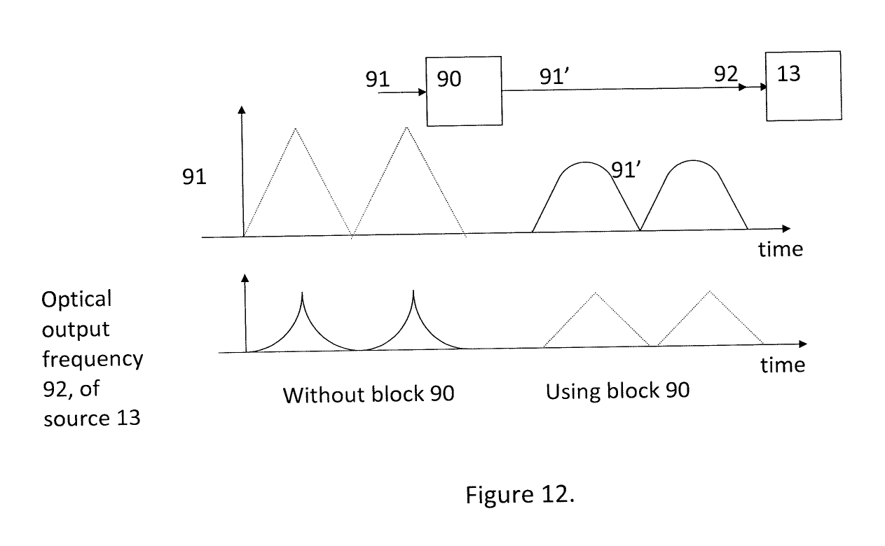

Let us suppose that the tuneable source 13 uses a tunable filter or is a low cost laser diode ramped in current, as reported in the paper by J. Zheng mentioned above. In this case, only a few nanometers tuning bandwidth is achievable, but sufficient to determine a depth resolution better than tens of microns. More expensive sources can be used to achieve micron resolution. In both cases above, either using a tuneable filter o a ramped laser diode, an electrical signal modifies the optical frequency of the output optical signal. In both cases, the output frequency manifests a nonlinear dependence on the electrical input signal. When tuning the source 13 in the embodiments in FIG. 4, 9, 10, the analogue signal applied to the tuning filter or the laser diode is altered in shape versus time. For instance let us say that the output frequency, 92, of source 13 varies faster than linearly as the controlling voltage increases. A simple nonlinear electric circuit, using diodes can be used to transform a triangle shaped voltage applied to the tuning filter or laser diode, into a nonlinear shape, approximated in FIG. 12 with a Gaussian, 91′. In this way, dependence of the frequency of the optical signal emitted by 13, versus time approaches linearity.

FIG. 12 shows the effect of the block 90 on the input voltage, 91 and on the output frequency, 92. For a linear ramp voltage shaped input, 91, the output, 91′, is nonlinear. Without block 90, if a linear ramp would be applied to 13, then the frequency 92 of the tuneable source 13 would be nonlinear, as shown by the lower inset left. Using the block 90, the frequency of the output optical signal, 92, becomes linear, as shown by the bottom right inset in FIG. 12.

### FD-LCI

Normally, the CCD (CMOS) arrays are read using shift registers which determine that data is shifted to the output linearly in time, pixel by pixel. Such arrays are read using a clock which feeds a shift register which controls the successive reading of pixels. Such clock is proposed here to be altered in time slots. By successively reducing or increasing the time slot of clock time interval, during the reading time TR (adjusted for quieter spectrum to 1/F as shown in FIG. 3B), the nonlinear spread of optical frequencies over the linear array is compensated for.

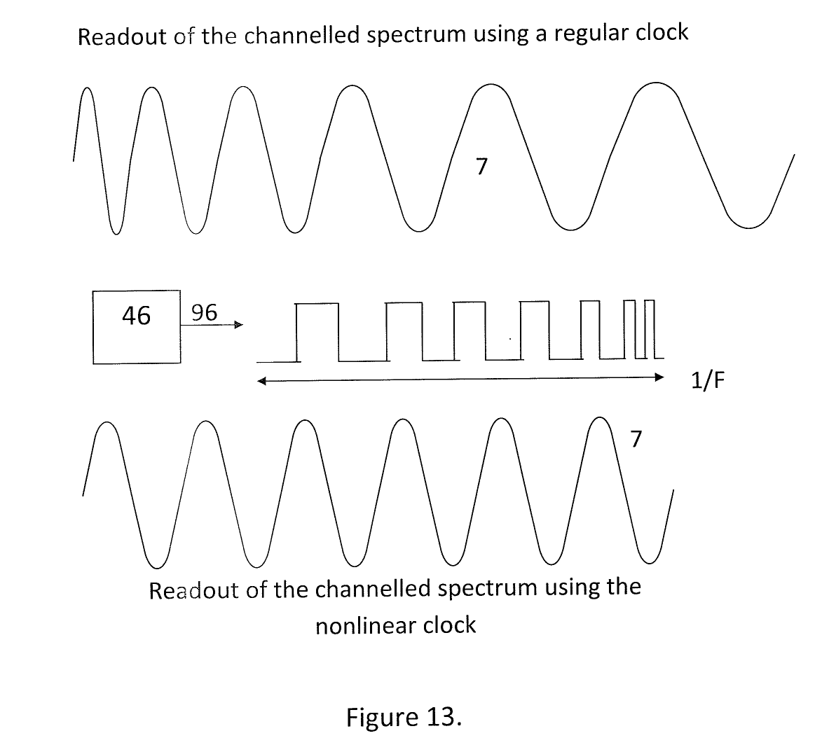

Let us consider a single reflector in the object, 3. FIG. 13 top illustrates the output signal, 7, which is a nonlinear sinusoidal signal with nonconstant repetition frequency due to nonlinear dependence of optical frequency along the photodetector array 63. As an example here, it is considered that from left to right, the period of the signal 7 increases. Then, a nonlinear clock generator, 46, can be used to alter the timing when each pixel in the array 63 is transferred out to signal 7, for the case shown in the top figure, by reducing the time interval the shift register is read from one cycle to the next. In case suc a nonlinear clock 96 is applied to the shift register of the array 63, the output of the array becomes a clear undistorted sinusoidal signal 7, whose spectrum presents a narrow well defined frequency component.

For each integration cycle, 1/F, pixel data is taken out using the nonlinear clock 96.

Obviously, the procedures above, as illustrated by FIGS. 12 and 13 are imperfect in terms of compensation of the nonlinear dependence of optical frequency versus time in the case of a swept source 13 or of the frequency spread of the signal output from the CCD array 63 in a FD-LCI set-up. The imperfection in the linearization of signal can be improved by increasing the number of diodes in the circuit 90.

The novel procedures according to the invention however help increasing the amplitude of the measuring signal 7 and in consequence, the sensitivity of the set-up. The larger the reference OPD value established for measurements, the better the improvement brought by applying the methods described in FIGS. 12 and 13. In case that the reference OPD is chosen at small values, then linearization of data is not necessary. In both cases in FIGS. 12 and 13, linearization of data versus optical frequency is performed by altering the speed of reading the channeled spectrum within each period, 1/F, of such reading. When using the linear array, the reading is altered by modifying the moment when signal from a given photosite is taken out within the scanning period. When using a swept source, the reading is altered by modifying the voltage shape which controls the moment a certain frequency is created, again, within the cycle 1/F.

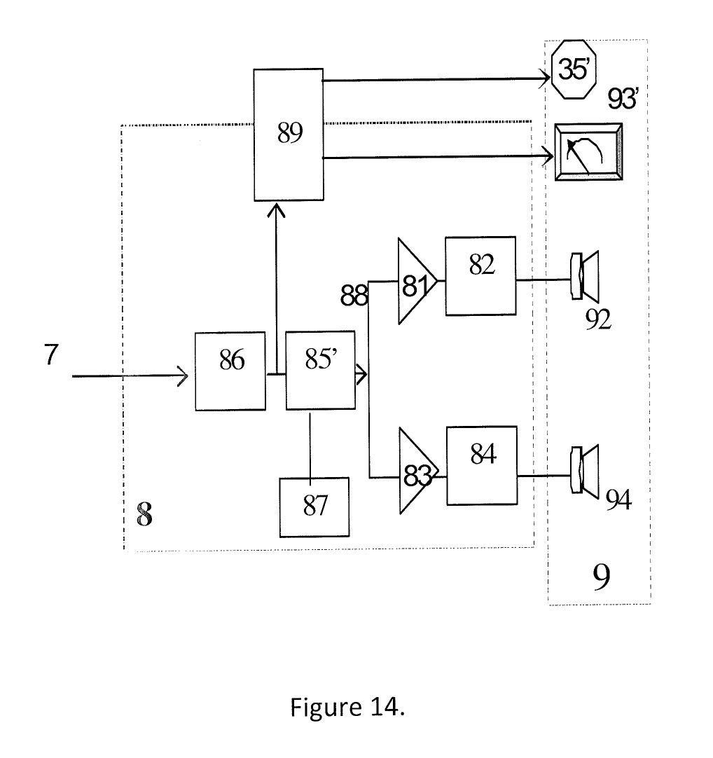

FIG. 14 presents an improved embodiment of the indicating block 8. All its building parts or only some can be electively used in any of the previous embodiments.

As a first improvement, because the channeled spectrum reading frequency F, may be in the audible range, a rejection filter, 86, is placed on the signal 7, before being sent to band pass filters 82 and 84.

As another possible improvement, the signal output of the rejection filter, 86, is beaten with a locally generated signal. A beating sound, 88, is produced by beating the signal 7 coming out of the interrogator 6 with a sinusoidal signal of a chosen frequency, G, generated by a signal generator 87, using a mixer 85′. For instance, let us consider that the reading of the channeled spectrum is performed at F=100 Hz and that the spectral interrogator, 6, has the resolution to read up to 200 peaks in the channeled spectrum. This leads to a range of frequencies for the signal 7 between F and 20 kHz, where the maximum corresponds to an OPD value of 2001_{c}. Let us say that the OPD value of interest, sought after, δz, is 201_{c} For this OPD value, the channeled spectrum contains 20 peaks and therefore the signal 7 oscillates at f_{ref}=2 kHz. The generator 87 is adjusted to G=f_{ref}. In this way, when the OPD is increased, the frequency of the signal out of 85′ decreases from 2 kHz to zero when OPD reaches the value of reference, δz, and increases again from zero to 20−2=18 kHz, if continuing to increase the OPD. In this way, the process of searching for the desired audio signal requires bringing the audio frequency to zero.

A more refined adjustment can now be devised. Because deciding exactly where the frequency of signal 88 is zero is affected by errors, the OPD value is adjusted by knobs 5 or 5′ until the frequency of signal 88 reaches, let us say 1 kHz, either side of the value shown by measuring means, 5, as E. The band pass filter 82 is tuned on 1 kHz and drives an LED or an analogue ammeter, and maxima are achieved for two positions of the adjusting means cvorresponding to values shown by the measuring means, 5, as E₁ and E₂. Let us say that OPD is continuously increased and E₁ corresponds to the first value of OPD when 1 kHz is obtained, corresponding to an OPD matching δz, then the frequency of signal 88 decreases, goes through zero and by continuing the increase in the OPD, the frequency of signal 88 increases from zero to 1 kHz at E₂(when the OPD is −δz). Then, the unknown length, E value can be determined more exactly, as (E₁+E₂)/2.

A frequency to amplitude converter, 89 could drive another needle meter, 95′, or a digital meter, or a display unit, 93′, where the colour suggests the strength of the signal. This is useful when the frequency of the channeled spectrum is higher than the audible range and when using the mixer 85′ which provides the same pitch, irrespective if the frequency of the signal 7 is lower or higher than the frequency of the signal generated by generator 87. The frequency to amplitude convertor 89 helps the user understand the absolute direction of OPD change while following the pitch of sound emitted by 94 after mixer 85′.

### Stethoscope

A related application is that of monitoring the movement of heart walls in organisms. Such organisms could be larvae, embryos, animals, humans. The beating rate can be easily translated into sound.

Let us consider that in a fly embryo, the heart, object 3 in the embodiments above, moves by 100 microns. Due to a limited number of pixels in the array 63, or limited number of frequency steps in the tuning of the frequency of the optical source, 13, the axial range of the FD-LCI system is limited. Let us say that the axial range is limited to 1 mm. For a coherence length of 10 microns, an OPD=1 mm will create 100 peaks in the channeled spectrum while the heart movement will correspond to a change in the number of peaks by 10. For a reading of the linear array 63 at 20 kHz (selected higher than the maximum audible frequency), the frequencies generated by reading the array 63 will be from 20 kHz to 2 MHz. The heart wall 3 may be at any OPD value within the 1 mm range, let us say that it is in the middle, at 0.5 mm, i.e. the frequency generated during the channeled spectrum reading is 1 MHz. A change in the OPD due to the heart wall movement will lead to a change of the frequency of 1 MHz by approximately 200 kHz (a change of 10 peaks, read at 20 kHz). Beating the photodetector array signal, 7, with a sinusoidal signal generated by 87, of F_{ref}=1 MHz, in the mixer 85′, will lead to a signal 88, pulsating at a frequency difference of 0 to 200 kHz. When fed to loudspeakers, 92 and 94, a variable pitch will be heard in 94, with frequencies from zero to 20 kHz, while 92 will deliver blips when the frequency coincides to that of the narrow band filter 82, with pauses followed again by the same succession of frequencies when the heart returns to the initial position. If the embryo 3 moves axially to a different settled axial position, by changing the value of F_{ref}, the new position is identified and monitoring can start again. The process of retuning F_{ref} is here equivalent to finding the new axial position of the gate in TD-LCI.

For enhanced sensitivity, the blocks 81 and 83 can be equipped with zero crossing circuits. Such circuits generate a narrow pulse anytime the incoming signal crosses the voltage value of zero. This can then be transformed into a sinusoidal signal with repetition frequency determined by the inverse of time interval from a zero crossing to the next. In this way, the frequency of the signal heard in 92 and 94 is strictly proportional to the repetition in the channeled spectrum and the signal amplitude does not depend on the amplitude of the channeled spectrum. Sometimes, without such zero crossing circuits, the amplitude of the channeled spectrum is so high that saturation may occur which will distort the sound produced by the loudspeakers 92 and 94. Irrespective of the amplitude of the incoming signal, the zero crossing sensing blocks 81, 83, will generate signals of constant amplitude and frequency depending on the repetition of the input signal only.

Obviously, for those skilled in the art, where splitters are in bulk, they could equally be in fiber and vice versa. Focusing elements throughout the disclosure could be curved mirrors or lenses.

Thus, it has been apparent that there has been provided, in accordance with the present invention an apparatus which fully satisfies the means, objects and advantages set forth hereinbefore.

Therefore, having described specific embodiments of the present invention, it will be understood that alternatives, modifications and variations thereof may be suggested by those skilled in the art.

1. Spectral interferometry apparatus for measuring an unknown length between a first point, A, and a second point, B, where point A is placed on the surface of an object, apparatus comprising:
an optical source;
an interferometer having at least two optical paths which provides an output optical signal;
a spectral interrogator arranged to deliver a measuring signal by interrogating the output optical signal;
an adjusting length device arranged to change the length of at least one of the optical paths of the interferometer, the adjusting length device having a measuring means providing a length measurement of the at least one optical path, and
a processing unit for the measuring signal, which provides an indicating signal to an indicating means;
wherein the spectral interferometry apparatus is arranged to perform the measurement of the unknown length by actuating the adjusting length device until a characteristic of the indicating signal provided by the indicating means reaches a desired value and the value of the unknown length is obtained from the indication of the measuring means.

2. Spectral interferometry apparatus according to claim 1, wherein the measuring means, is a micrometer screw, a graded knob or a sliding ruler.

3. Spectral interferometry apparatus according to claim 1, wherein
the optical source is broadband; and
the spectral interrogator comprises a spectrometer equipped with a linear photodetector array scanned at a frequency F to provide the measuring signal.

4. Spectral interferometry apparatus according to claim 1, wherein the optical source is a narrow band swept source, and the spectral interrogator is equipped with a photodetecting unit which provides the measuring signal while tuning the optical source at rate F.

5. Spectral interferometry apparatus according to claim 1, wherein the indicating means contains a first loudspeaker or a earphone and the characteristic of the said indicating signal is the intensity of the sound emitted by the loudspeaker or the earphone.

6. Spectral interferometry apparatus according to claim 1, wherein the indicating means contains an additional second loudspeaker and the characteristic of the said indicating signal is the pitch of the sound emitted by the loudspeaker.

7. Spectral interferometry apparatus according to claim 1, wherein the indicating means contain a needle or digital meter and the characteristic of the said indicating signal is the voltage or current indicated by the meter.

8. Spectral interferometry apparatus according to claim 1, wherein the indicating means contains a luminous emitter and the characteristic of the said indicating signal is the intensity of the light emitted by the luminous emitter or its colour.

9. Spectral interferometry apparatus according to claim 1 where the electronic processing unit is equipped with at least a band-pass filter which is tuned on a frequency within the audible range delivered to the said loudspeakers.

10. Spectral interferometry apparatus according to claim 1 where the electronic processing unit is equipped with a frequency to amplitude convertor which translates the frequency of the said measuring signal into an amplitude for the indicating signal.

11. Spectral interferometry apparatus according to claim 3, where the linear photodetector array is read using a clock pulse with variable time interval between successive pulses and where the time interval between clock pulses is altered in such a way as to produce a linear dependence between the moment of time a pixel is read and the optical frequency in the said channeled spectrum.

12. Spectral interferometry apparatus according to claim 4, where the said swept source is controlled by a voltage source, wherein the voltage source is arranged to produce a voltage signal that during the tuning cycle 1/F has a voltage versus time variation distorted in such a way so that the optical frequency of the swept source varies linearly in time.

13. Spectral interferometry apparatus according to claim 1 further comprising: a 1^{st} splitter to split light from the optical source to an object arm and into a reference arm where the first arm conveys light to point A via an object interface optics, and back from A via the object interface optics and 1^{st} splitter towards the spectral interrogator and light along the reference arm is also sent to the spectral interrogator where it superposes with the object beam, and where the path travelled by the object beam from the 1^{st} splitter to the object and back to the interrogator defines an object path and the path traveled by the reference beam from the 1^{st} splitter towards the interrogator defines a reference path and where the adjusting length device alters the optical path difference between the object and reference paths.

14. Spectral interferometry apparatus according to claim 1, wherein the said 2^{nd} point B is placed inside the apparatus, so that the unknown length refers to the distance between the object, point A, and the apparatus, and the reference beam is circulated inside the apparatus.

15. Spectral interferometry apparatus according to claim 1, wherein the adjusting length device is a translation stage, equipped with a knob or a cursor, where the translation stage is moved by actuating on the knob or the cursor and the knob or the cursor are marked with divisions or the stage is equipped with a ruler, and where the positions of the stage can be identified from the divisions of the knob, cursor or ruler, and where the translation stage carries elements parts of the interferometer which determine a change in the optical path traveled by light.

16. Spectral interferometry apparatus claim 1 wherein point B is the bottom part of an object and the unknown distance is the thickness, E of the object measured between A and B.

17. Spectral interferometry apparatus according to claim 16, wherein the said object is the eye and the unknown length, E, is the eye length.

18. Spectral interferometry apparatus according to claim 17, comprising: a 1^{st} splitter to split light from the optical source to an object arm and into a reference arm where the first arm conveys light to point A via an object interface optics, and a 2^{nd} splitter, and back from point A, via second splitter and object interface optics and back to 1^{st} splitter towards the spectral interrogator and light along the reference arm is sent via a reference interface optics, via the 2^{nd} splitter, to the eye, and where the reference interface optics essentially focuses the reference beam to the retina, acting as point B, and the light returned from B is sent via the eye, 2^{nd} splitter, reference interface optics to the spectral interrogator where it superposes with the object beam, and where the path traveled by the object beam from the 1^{st} splitter to A and back to the interrogator defines an object path and the path traveled by the reference beam from the 1^{st} splitter towards B and back and then to the spectral interrogator defines a reference path and where the adjusting length device alters the optical path difference between the object and reference paths.

19. Spectral interferometry apparatus according to claim 16, where the light source is connected to input 1 of a 2×2 splitter, with two inputs and two outputs, where light from the 1^{st} output is sent via an interface optics towards point A and point B, where the interface optics has sufficient long depth of focus to collect returned signals from both A and B back via the interface optics, to the 1^{st} output of the 2×2 splitter, which delivers the superposition of the two beams from A, and B, to the 2^{nd} input of the 2×2 splitter, which conveys the light to an adjustable two beam interferometer, whose optical path difference measured between its path lengths is OPD₂, and where the said adjusting length device in claim 1 is used to alter OPD₂ and where the said output optical signal from the interferometer in claim 1 is made from the two reflected beams from point A and point B, whose optical path difference is the object thickness, OPD₁=E and where the said spectral interrogator produces a 1^{st} measuring signal of frequency f₁ clue to E and produces a 2^{nd} measuring signal of frequency f₂ due to OPD₂ and where the processing unit contains an electrical mixer which mixes the 1^{st} and the 2^{nd} measuring signals to provide a 3^{rd} measuring signal of frequency DF which is sent to the said indicating means.

20. Spectral interferometry apparatus according to claim 19, wherein the spectrometer uses an analogue linear photodetector array read at a frequency F where the frequency F is larger than 18 kHz and the frequency of the 3^{rd} measuring signal, DF, represents the characteristic of the output signal and is within the audible range.

21. Spectral interferometry apparatus according to claim 3, wherein the processing unit contains: an amplifier of the measuring signal, a rejection filter tuned on the frequency F, and a band-pass filter tuned on reference frequency within the audible range.

22. Spectral interferometry apparatus according to 1 where the indicating means is provided with indicators of sufficient signal being returned from the two measuring points, A and B, further comprising the object interface optics and reference interface optics equipped with focus adjusting elements for actuation to enhance the strength of the signal being returned from the two measuring points A and B.

23. Spectral interferometry method for measuring an unknown length between a first point, A, and a second point, B, including adjusting the optical path difference (OPD) of an interferometer until a certain characteristic of an indicating signal reaches a desired value, where the OPD is adjusted actuating on measuring means equipping adjusting means and where the unknown length is inferred from the indication of the measuring means when the characteristic of the indicating signal has reached the desired value.

24. Spectral interferometry method according to claim 23 where the indicating signal is sound and the desired value is the maximum sound.

25. Spectral interferometry method according to claim 23 where the indicating signal is sound and the desired value is the pitch of the sound emitted.
